(19) United States
(12) Patent Application Publication
(10) Pub. No.: US 2020/0329964 A1
(43) Pub. Date: Oct. 22, 2020

(54) Image Processing Method and System Using the Same

(71) Applicant: FaceHeart Inc., Hsinchu City (TW)

(72) Inventors: Tsuey-Huey Shiue, Hsinchu City (TW); Yi-Chiao Wu, New Taipei City (TW); Wen-Chung Chen, Taoyuan City (TW); Kuan-Hung Chen, Hsinchu City (TW)

(21) Appl. No.: 16/460,998

(22) Filed: Jul. 2, 2019

(30) Foreign Application Priority Data
Apr. 18, 2019 (TW) 108113537

(51) Int. Cl. A61B 5/00; G06T 7/00

(52) U.S. Cl. CPC A61B 5/004; A61B 5/0035; A61B 5/0037; G06T 7/0012

(57) ABSTRACT

An image processing method for image-based physiological measurement, includes converting at least one user's image signal into image data; determining at least one region of interest within the image data; analyzing image information inside the region of interest to generate physiological information of the user; determining a feedback control signal or a control signal to optimize the physiological information of the user; and adjusting an image sensing unit or an image signal processing unit according to the feedback control signal or the control signal.

### BACKGROUND OF THE INVENTION

### 1. Field of the Invention

The present invention relates to an image processing method and system using the same, and more particularly, to an image processing method and system for measuring physiological information.

### 2. Description of the Prior Art

Image-based physiological signal detection methods have been proposed for a period. Related techniques such as “remote plethysmographic imaging using ambient light” by Wim Verkruysse, and “algorithmic Principles of Remote-PPG” by Wenjin Wang, have been widely tested to verify the possibility of using remote-PPG (as rPPG) for the heart rate measurements. The rPPG method can be further used for other physiological signal measurement including the heart rate variability (HRV), Blood Pressure, Respiration Rate, and so on.

All of these methods apply a camera to take image pictures or video for analysis. A legacy camera usually includes at least an image sensor to convert the light signal into a raw image data, and an image signal processor (ISP) to adjust and optimize the image quality automatically for human vision system (HVS) . An optimization process might be utilized for adjusting brightness, contrast, color balance, or even performing time-space interpolation of the image data. Additional image compression/decompression, noise reduction, or edge enhancement schemes might be also included. In addition, an analyzer and a processor of the above physiological signal detection methods process the adjusted images after the image signal processor.

However, the feeble physiological information hidden in the images are easily distorted or suppressed during the optimization processes of the ISP. To keep the desired physiological information as complete and accurate as possible, an image signal processing method and architecture for physiological information measurement is required.

### SUMMARY OF THE INVENTION

Therefore, the present invention provides an image signal processing method and system using the same capable of optimizing the image signal quality of the physiological information quality and accuracy within the image data, instead of optimizing the image quality for the human vision system.

An embodiment of the present invention discloses an image processing method for image-based physiological measurement, comprising converting at least one user's image signal into image data; determining at least one region of interest within the image data; analyzing image information inside the region of interest to generate physiological information of the user; determining a feedback control signal or a control signal to optimize the physiological information of the user; and adjusting an image sensing unit or an image signal processing unit according to the feedback control signal or the control signal.

Another embodiment of the present invention discloses an image system for image-based physiological measurement, comprising an image sensing unit, configured to convert light image into raw image data; an image signal processing unit, configured to perform a plurality of image signal adjustment functions on the raw image data, and to generate image data; a region of interest detecting unit, configured to detect whether a pre-defined ROI pattern exists within the image data, and to provide a position of the pre-defined ROI pattern within the image data; and a physiological signal processing unit, configured to analyze the image data within the pre-defined ROI pattern, to generate at least one physiological information, and to provide a control signal or a feedback control signal to the image processing unit or the image sensing unit.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIGS. 1-11 are schematic diagrams of an image processing system according to an embodiment of the present invention.

FIG. 12 is a flowchart diagram of an image processing process according to an embodiment of the present invention.

### DETAILED DESCRIPTION

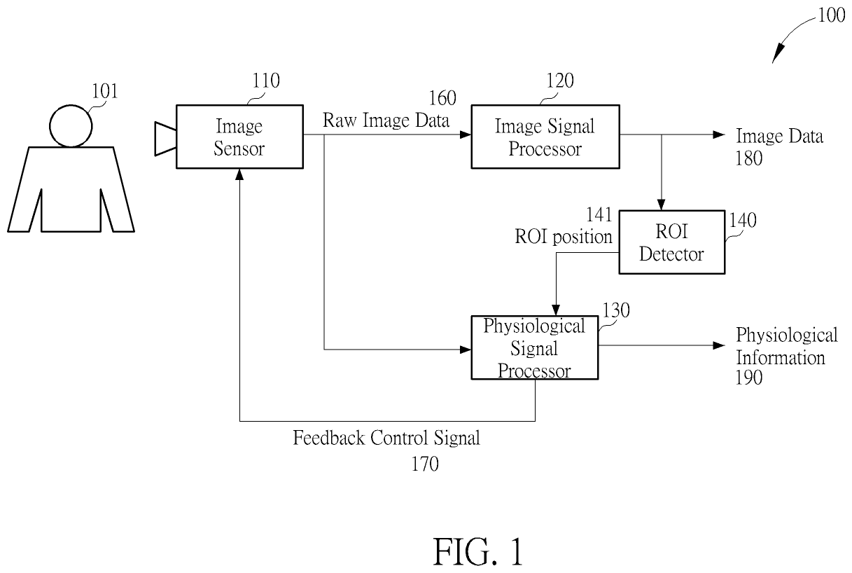

Please refer to FIG. 1, which is a schematic diagram of an image processing system 100 according to an embodiment of the present invention. The image processing system 100 includes an image sensor 110 to convert at least one user's image 101 into a raw image data 160. An image signal processor 120 is configured to receive the raw image data 160 to perform automatic adjustment functions, such as Auto White Balance (AWB), Auto Exposure (AE), Auto Focus (AF), Gamma Correction, Edge Enhancement (EE), Hue and Saturation adjustment, Noise Reduction (NR), etc., and output to following stages, e.g. display or storage device. A region of Interest (ROI) detector is configured to receive the adjusted image data 180 from the image signal processor 120, to detect any pre-defined ROI pattern existed within the image data 180, and to deliver a coordination of an ROI position 141. A physiological signal processor 130 receives the raw image data 160 as well as the ROI position 141, to analyze the image within the ROI region, and to generate at least one physiological information 190 after signal processing and calculations. The physiological signal processor 130 may generate a feedback control signal 170 to the image sensor 110, to change a plurality of image sensor settings, in order to optimize or to enhance the physiological information quality within the raw image data.

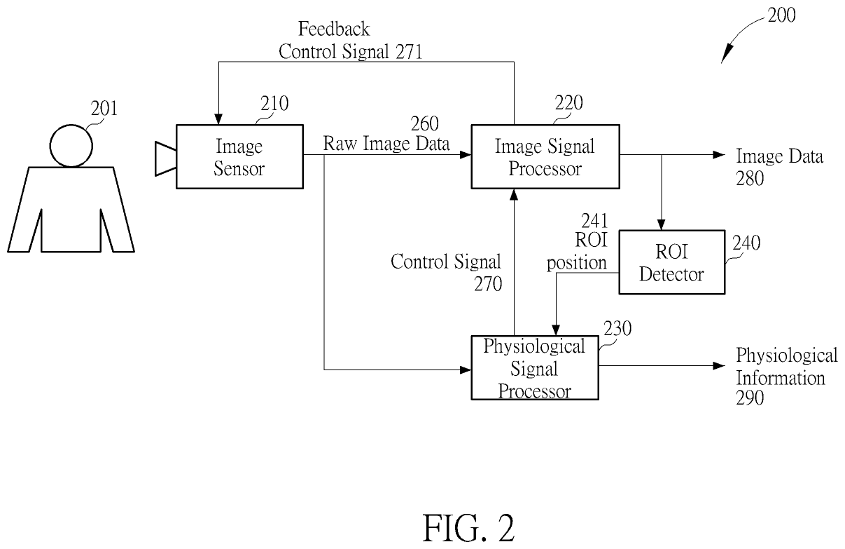

FIG. 2 is a schematic diagram of an alternative image processing system 200 according to an embodiment of the present invention. The image processing system 200 includes a physiological signal processor 230 to receive the raw image data 260 and an ROI position 241 similar to the image processing system 100, to analyze the image within the ROI region, and to generate at least one physiological information 290 after the signal processing and calculations. Instead of sending the feedback control signal 170 directly to the image sensor 110 from the physiological signal processor 130 as shown in FIG. 1, a control signal 270 of the image processing system 200 is generated and sent to the image signal processor 220 from the physiological signal processor 230 based on analysis results of the physiological information 290 so as to determine optimization settings for both of the image signal processor 220 and the image sensor 210, and to improve the information quality of the physiological information 290. The image signal processor 220 sends out the feedback control signal 271 to the image sensor 210. The feedback control signal 271 includes the optimize settings generated by either the physiological signal processor 230 and/or the image signal processor 220.

Parameters of the optimization settings maybe adjusted within the image sensor 110 or 210 and may include but not limited to the following items: image size and resolution, video frame rate, shutter speed or exposure time, exposure compensation, ISO, focus or focal length, aperture, analog gain, digital gain, gain on each color channel, sensitivity, high dynamic range (HDR) settings, black level calibration, and so on.

Functions of the image signal processor 120 or 220 may include but not limited to the following items: Auto White Balance (AWB), Auto Exposure (AE), Auto Focus (AF), Gamma Correction, Edge Enhancement (EE) , Hue and Saturation adjustment, and Noise Reduction (NR), and so on.

The physiological information 190 or 290 may be generated by the physiological signal processor 130 or 230 and may include but not limited to the following items: Heart Rate, Respiration Rate, Blood Pressure, Oxygen Saturation, Blood Sugar Level, Body Temperature, Photoplethysmography(PPG), remote Photoplethysmography(rPPG), and so on.

The Region of Interest (ROI) detector 140 or 240 may detect at least one position of the following regions: face, neck, chest, palm, arm, or skin of the rest part of the body, and so on.

The physiological signal processor 130 or 230 not only generates the physiological information 190 or 290, but also analyzes and calculates the image data to find out other optimization methods to change the settings or configurations of the image sensor 110/210 and/or image signal processor 120/220, so as to enhance quality and accuracy of the physiological information 190/290.

As an example, the image sensor 110/210 may be adjusted for an optimization purpose with the following items, but not limited thereto:
Adjust image size and resolution to meet the target size.
Adjust the video frame rate to improve the video quality.
Adjust the shutter speed to prevent from over/underexposure.
Adjust the focal length to sharpen the image.
Adjust the analog gain, digital gain, aperture and ISO to prevent from over/under-exposure.

As an example, the image signal processor 120/220 may be adjusted or configured for the optimization purpose with the following items, but not limited thereto:
Adjust Auto Exposure (AE) to keep the maximum value of each pixel under a certain level (e.g., 200) and keep the minimum value of each pixel above a certain level (e.g., 50) to prevent from distorting the physiological information.
Adjust Auto white Balance (AWB) to keep the color channel ratio in a manner from distorting the physiological information
Adjust Gamma Correction, Hue and Saturation to keep the image color ratio from being distorted.
Adjust Edge Enhancement (EE) and Auto Focus (AF) to keep the clear images.
Adjust Noise Reduction (NR) to reduce the noise within the images.

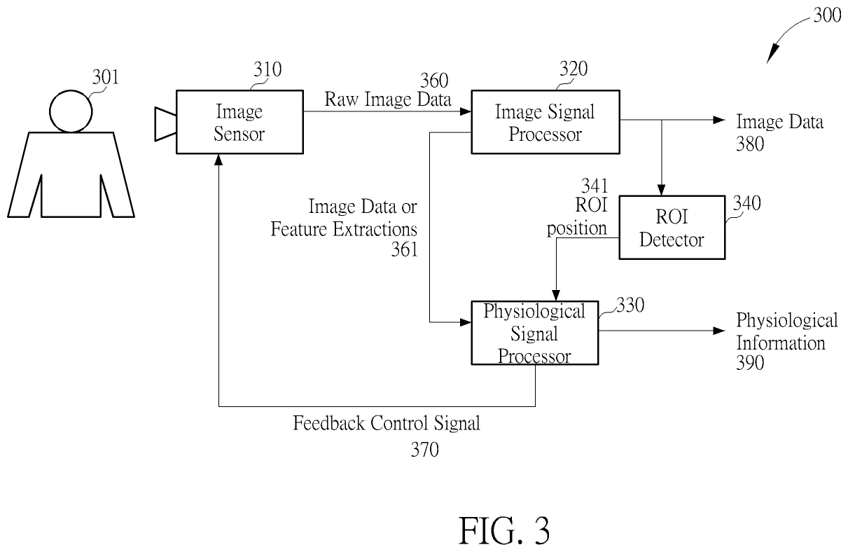

FIG. 3 is a schematic diagram of an alternative image processing system 300 according to an embodiment of the present invention. Instead of receiving the raw image data 160 from the image sensor 110 by the physiological signal processor 130 in FIG. 1, the physiological signal processor 330 in FIG. 3 is configured to receive the image data or feature extractions 361 from the image signal processor 320 to perform the physiological signal analysis and calculations, and to generate a feedback control signal 370 for the image sensor 310.

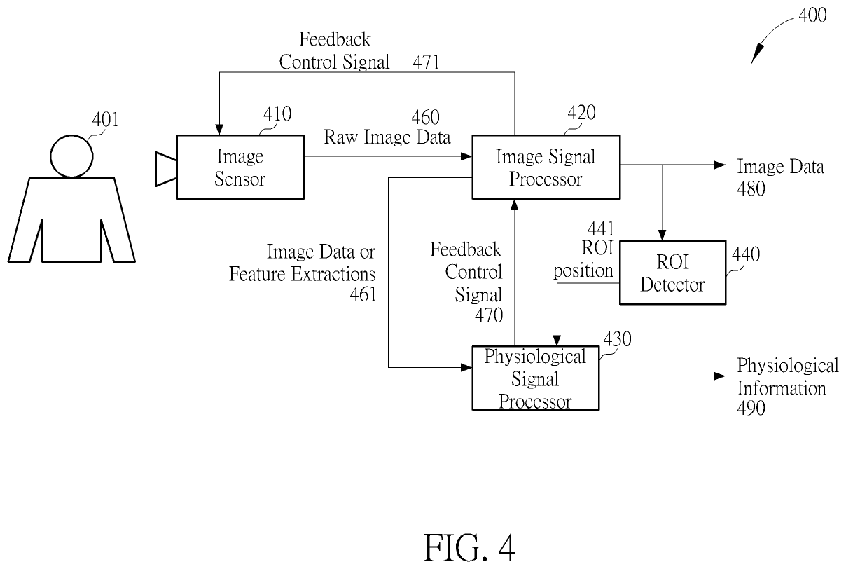

FIG. 4 is a schematic diagram of an alternative image processing system 400 according to an embodiment of the present invention. Different with FIG. 3, a control signal 470 is generated and sent to the image signal processor 420 from the physiological signal processor 430 based on the analysis results of the physiological information 490, so as to determine the optimization settings for both of the image signal processor 420 and the image sensor 410 and to improve the information quality of the physiological information 490. The image signal processor 420 is configured to send out the feedback control signal 471 to the image sensor 410. The feedback control signal 471 includes the optimize settings for the image sensor 410 generated by either the physiological signal processor 430 and/or the image signal processor 420.

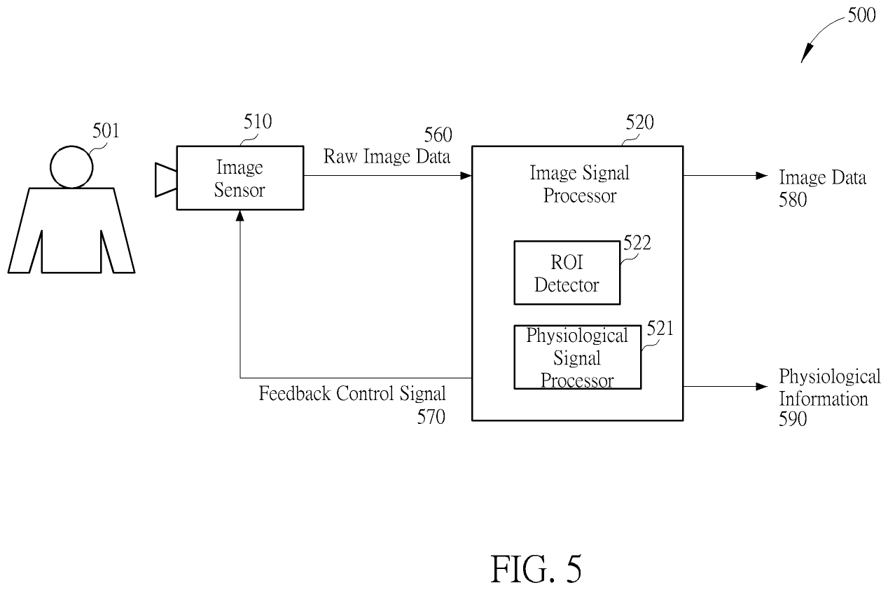

FIG. 5 is a schematic diagram of an alternative image processing system 500 according to an embodiment of the present invention. Both of the ROI detector 522 and the physiological signal processor 521 are integrated with the image signal processor 520. The image sensor 510 is configured to convert at least one user's image 501 into raw image data 560. The image signal processor 520 is configured to receive the raw image data 560, so as to perform automatic image adjustment functions. The ROI detector 522 is configured to detect whether any pre-defined ROI pattern exists within the raw image data, and to analyze the image data within the ROI region by the physiological signal processor 521, so as to generate at least one physiological information 590. In addition, a feedback control signal 570 is generated and sent to the image sensor 510 to change the image sensor settings for the physiological information quality optimization and enhancement within the raw image data.

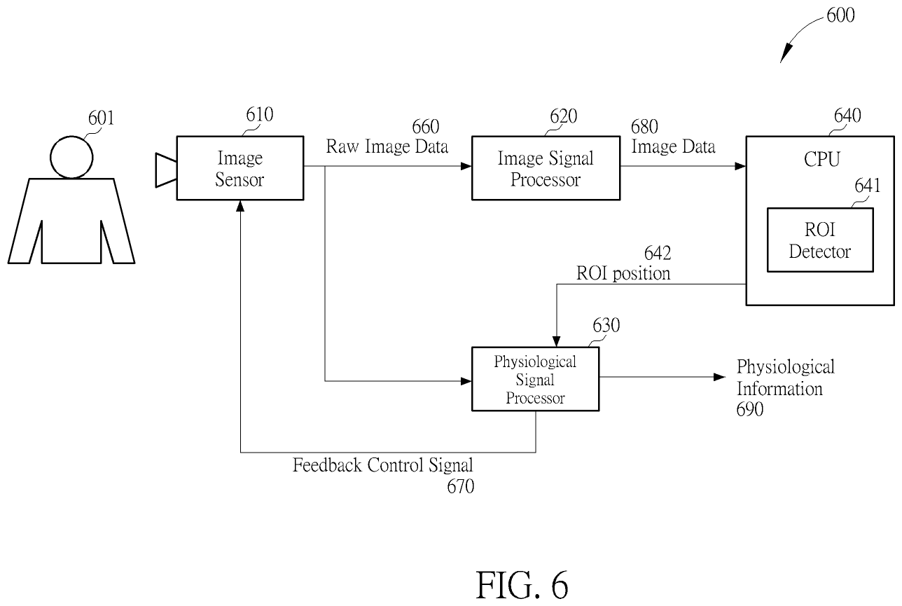

FIG. 6 is a schematic diagram of an alternative image processing system 600 according to an embodiment of the present invention. A central processing unit (CPU) 640 is added in FIG. 6, and the ROI detector 641 is integrated with the CPU 640. Image data 680 from the image signal processor 620 is generated and sent to the CPU 640, which may be utilized by user interface applications and so on. The ROI detector 641 is configured to detect whether any pre-defined ROI pattern exists within the image data 680, and to deliver the coordination of the ROI position 642 to the physiological signal processor 630. Then, a feedback control signal 670 is sent to the image sensor 610 from the physiological signal processor 630 to change the image sensor settings for the physiological information quality optimization and enhancement within the raw image data.

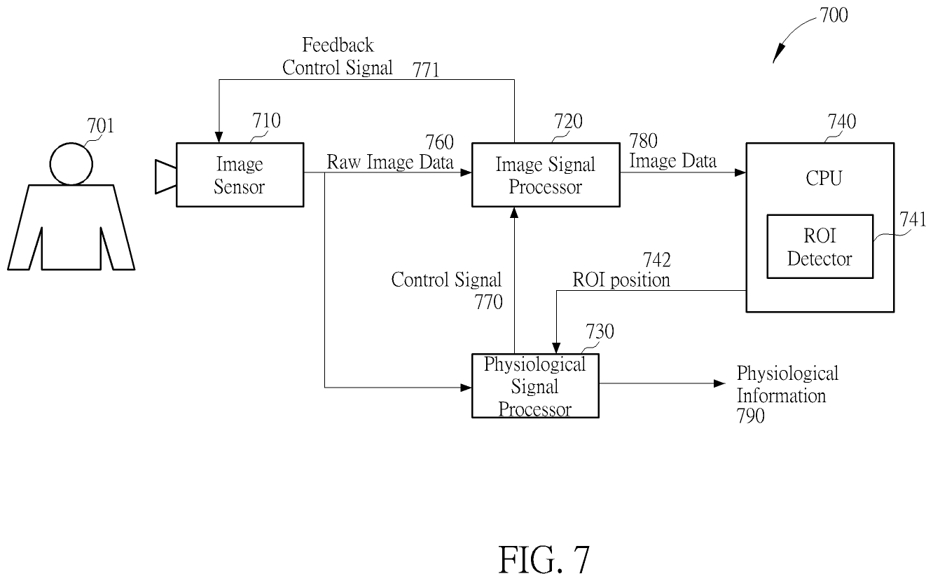

FIG. 7 is a schematic diagram of an alternative image processing system 700 according to an embodiment of the present invention. A control signal 770 is generated and sent to the image signal processor 720 from the physiological signal processor 730 based on the analysis results of the physiological information 790 to determine the optimization settings for both of the image signal processor 720 and the image sensor 710 to improve the information quality of the physiological information 790. Then, the image signal processor 720 is configured to send out the feedback control signal 771 to the image sensor 710. The feedback control signal 771 includes the optimize settings for the image sensor 710 generated by either the physiological signal processor 730 and/or the image signal processor 720.

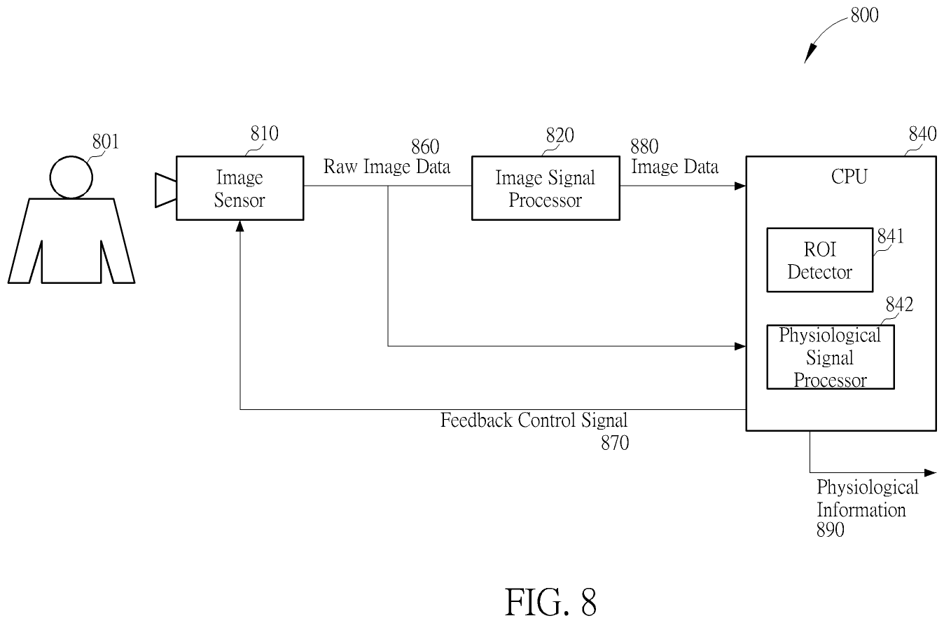

FIG. 8 is a schematic diagram of an alternative image processing system 800 according to an embodiment of the present invention. Both of the ROI detector 841 and the physiological signal processor 842 are integrated with the CPU 840. The image data 880 is generated by the image signal processor 820 as well as the raw image data 860 from the image sensor 810, which are sent to the CPU 840. The ROI detector 841 is configured to calculate the coordination of the ROI position, which is sent to the physiological signal processor 842. The CPU 840 is configured to output the physiological information 890 for further applications. A feedback control signal 870 is provided to the image sensor 810 in order to change some of the image sensor settings for the physiological information quality optimization and enhancement.

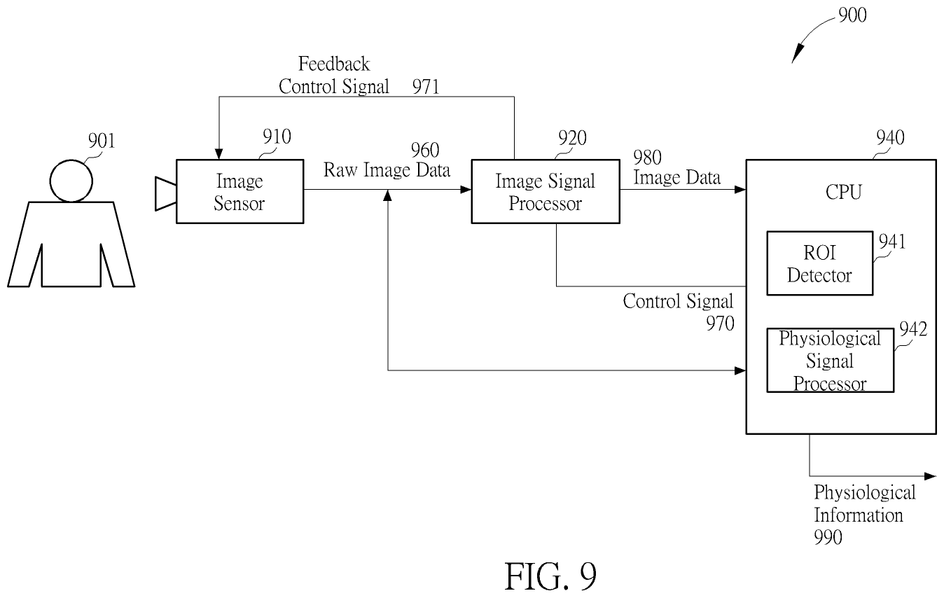

FIG. 9 is a schematic diagram of an alternative image processing system 900 according to an embodiment of the present invention. A control signal 970 is generated and sent to the image signal processor 920 from the CPU 940, based on the analysis results of the physiological information 990 to determine the optimization settings for both of the image signal processor 920 and the image sensor 910 so as to improve the information quality of the physiological information 990. The image signal processor 920 is configured to send out the feedback control signal 971 to the image sensor 910. The feedback control signal 971 includes the optimize settings for the image sensor 910 generated by either the CPU 940 and/or the image signal processor 920.

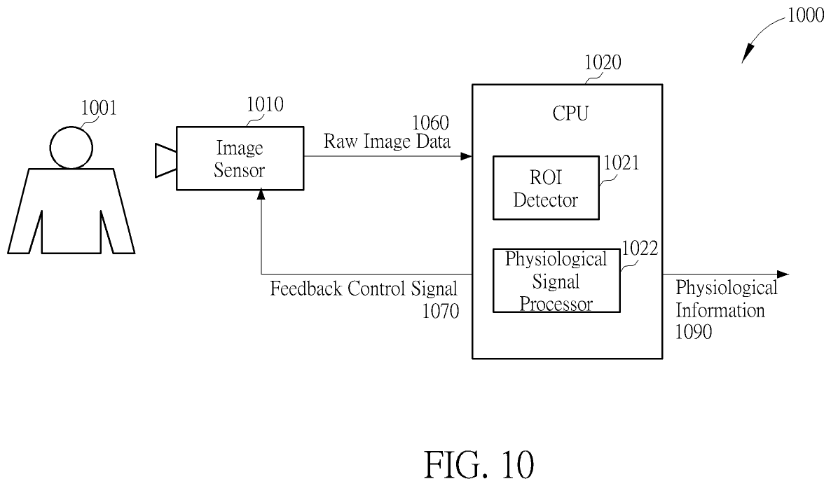

FIG. 10 is a schematic diagram of an alternative image processing system 1000 according to an embodiment of the present invention. In FIG. 10, the image processing system 1000 is an alternative structure of the image processing system 500 of FIG. 5. Both of the ROI detector 1021 and the physiological signal processor 1022 are integrated with the CPU 1020. The image sensor 1010 is configured to convert at least one user's image 1001 into a raw image data 1060. The CPU 1020 is configured to receive the raw image data 1060. And, the internal ROI detector 1021 is configured to detect whether any pre-defined ROI pattern within the raw image data. The image data within the ROI region is analyzed by the physiological signal processor 1022 to generate at least one physiological information 1090. The CPU 1020 is configured to generate a feedback control signal 1070 to the image sensor 1010 to change the image sensor settings for the physiological information quality optimization and enhancement within the raw image data.

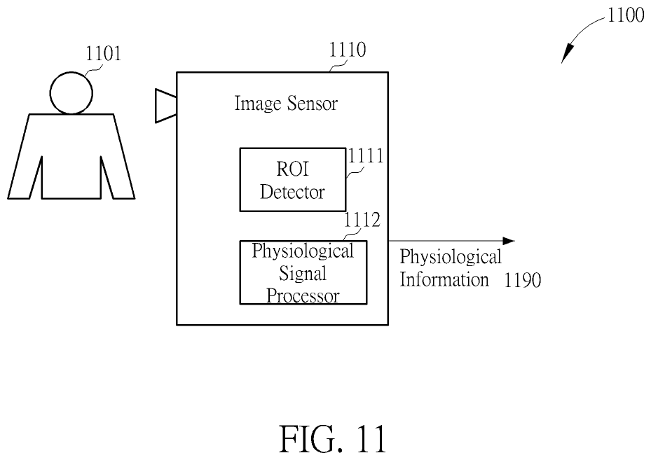

FIG. 11 is a schematic diagram of an alternative image processing system 1100 according to an embodiment of the present invention. The image processing system 1100 in FIG. 11 is an alternative image processing system of the image processing system 1000 in FIG. 10. Both the ROI detector 1111 and the physiological signal processor 1112 are directly integrated with the image sensor 1110. The image sensor 1010 is configured to convert at least one user's image 1001 into image data format. The internal ROI detector 1111 is configured to detect whether any pre-defined ROI pattern exists within the image data. The internal physiological signal processor 1112 is configured to analyze the image data within the ROI region and output at least one physiological information 1090. The image sensor 1110 may be adjusted based on the physiological information quality optimization and enhancement purposes.

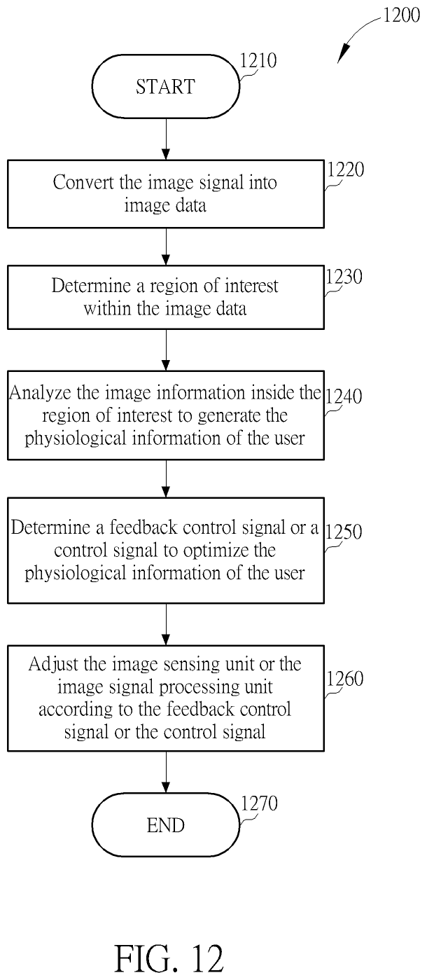

A flowchart of the present invention may be summarized to an image processing process 1200 as shown in FIG. 12. The image processing process 1200 includes the following steps:

Step 1210: Start.

Step 1220: Convert the image signal into image data.

Step 1230: Determine a region of interest within the image data.

Step 1240: Analyze the image information inside the region of interest to generate the physiological information of the user.

Step 1250: Determine a feedback control signal or a control signal to optimize the physiological information of the user.

Step 1260: Adjust the image sensing unit or the image signal processing unit according to the feedback control signal or the control signal.

Step 1270: End.

In summary, the present invention provides an image processing method and system for image-based physiological measurement capable of preventing damages to the image data adjusted by the image signal sensor or the image signal processor, which affects the feature extractions of the image, and improving the stability of the physiological information of the image data.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. An image processing method for image-based physiological measurement, comprising:
converting at least one user's image signal into image data;
determining at least one region of interest within the image data;
analyzing image information inside the region of interest to generate physiological information of the user;
determining a feedback control signal or a control signal to optimize the physiological information of the user; and
adjusting an image sensing unit or an image signal processing unit according to the feedback control signal or the control signal.

2. The image processing method of claim 1, wherein the physiological information comprises at least one of Heart Rate, Respiration Rate, Blood Pressure, Oxygen Saturation, Blood Sugar Level, Body Temperature, Photoplethysmography (PPG)and remote Photoplethysmography (rPPG).

3. The image processing method of claim 1, wherein the region of Interest comprises at least one of face, neck, chest, palm, arm, and skin of the body.

4. The image processing method of claim 1, wherein a plurality of functions of the image sensing unit are adjusted by the control signal or the feedback control signal, which comprises at least one of an image size, an image resolution, a video frame rate, a shutter speed, an exposure time, an exposure compensation, an ISO, a focal length, an aperture, an analog gain, a digital gain, a gain on each color channel, a sensitivity, a high dynamic range (HDR) setting, and a black level calibration.

5. The image processing method of claim 1, wherein a plurality of functions of the image signal processing unit are adjusted by the control signal or the feedback control signal, which comprises at least one of white balance, exposure, focus, gamma correction, edge enhancement, hue, saturation, and noise reduction.

6. An image system for image-based physiological measurement, comprising:
an image sensing unit, configured to convert light image into raw image data;
an image signal processing unit, configured to perform a plurality of image signal adjustment functions on the raw image data, and to generate image data;
a region of interest detecting unit, configured to detect whether a pre-defined ROI pattern exists within the image data, and to provide a position of the pre-defined ROI pattern within the image data; and
a physiological signal processing unit, configured to analyze the image data within the pre-defined ROI pattern, to generate at least one physiological information, and to provide a control signal or a feedback control signal to the image processing unit or the image sensing unit.

7. The image system of claim 6, wherein the image data analyzed by the physiological signal processing unit is from the raw image data after the image sensing unit, or the image data after the image signal processing unit.

8. The image system of claim 6, wherein the image signal processing unit is configured to perform the plurality of image signal adjustment functions on the raw image data, and to generate a set of feature extractions; the physiological signal processing unit is configured to analyze the set of feature extractions.

9. The image system of claim 6, further comprising:
a central processing unit (CPU) , configured to perform a plurality of additional signal processing or control functions.

10. The image system of claim 9, wherein the central processing unit is configured to integrate with the region of interest detecting unit to detect the pre-defined ROI pattern.

11. The image system of claim 9, wherein the central processing unit is configured to integrate with the image signal processing unit to perform the plurality of image signal adjustment functions.

12. The image system of claim 9, wherein the central processing unit is configured to integrate with the physiological signal processing unit to analyze the image data within the pre-defined ROI pattern.

13. The image system of claim 6, wherein the image sensing unit is configured to integrate with the image signal processing unit to perform the plurality of image signal adjustment functions.

14. The image system of claim 6, wherein the image sensing unit is configured to integrate with the region of interest detecting unit to detect the pre-defined ROI pattern.

15. The image system of claim 6, wherein the image sensing unit is configured to integrate with the physiological signal processing unit to analyze the image data within the pre-defined ROI pattern.

16. The image system of claim 6, wherein the image signal processing unit is configured to integrate with the region of interest detecting unit to detect the pre-defined ROI pattern.

17. The image system of claim 6, wherein the image signal processing unit is configured to integrate with the physiological signal processing unit to analyze the image data within the pre-defined ROI pattern.
